(19) United States
(12) Patent Application Publication
(10) Pub. No.: US 2006/0099192 A1
(43) Pub. Date: May 11, 2006

(54) Method for generating an expandable tissue culture from progenitor cells and tissue so generated

(75) Inventor: Horst Peschel, Gummersbach (DE)

Correspondence Address: AKIN GUMP STRAUSS HAUER & FELD L.L.P., ONE COMMERCE SQUARE, 2005 MARKET STREET, SUITE 2200, PHILADELPHIA, PA, 19103, US

(73) Assignee: NeuroProgen GmbH Leipzig

(21) Appl. No.: 11/280,482

(22) Filed: Nov. 16, 2005

Related U.S. Application Data
(62) Division of application No. 09/596,507, filed on Jun. 19, 2000, now abandoned.

(30) Foreign Application Priority Data
Jun. 19, 1999 (DE) 199 28 210.2

(51) Int. Cl. C12N 5/08; A61K 35/30; A61K 48/00

(52) U.S. Cl. 424/93.7; 435/325; 435/368; 424/93.21; 435/455

(57) ABSTRACT

This invention relates to neuronal tissue which is suitable to restore neuronal deficits following transplantation. To reduce immunological side effects and to increase the microbiological and genetic safety of the tissue we propose tissue that does not contain glial cells and is maintained in vitro for prolonged periods. The method to generate expandable determined neuronal progenitor cell cultures includes the following procedures: dissection of appropriate mammalian brain regions, isolation of progenitor cells, expansion of progenitor cells, selection and expansion of individual cells and priming.

### CROSS-REFERENCE TO RELATED APPLICATIONS

This application is a divisional application of U.S. patent application Ser. No. 09/596,507, filed Jun. 16, 2000, which is hereby incorporated by reference herein, in its entirety, for all purposes.

### BACKGROUND OF THE INVENTION

This invention relates to brain tissue, which is developed from immature neuronal precursor cells as a source for tissue transplantation in neurological and neurosurgical disorders and the method of generating such tissue.

Restorative treatment strategies have been have been exploited in respect to many neurological and neurosurgical disorders. The underlying idea is to replace dead or non-functional tissue by appropriate cell suspensions. There is a chance to treat, e.g. Parkinson's disease with implants consisting of dopaminergic neurons, Alzheimer's disease with cholinergic neurons, Huntington's disease with striatal GABAergic neurons, and multiple system atrophy with dopaminergic and GABAeric neurons.

Unfortunately, these treatment options are limited by the lack of appropriate tissue, which is derived from human embryos. In addition, there is debate about the need for immunosuppressive therapy following transplantation. Most current protocols involve immunosuppression causing significant side effects. Tissue derived from human embryos also needs to be transplanted into patients within a few days severely limiting microbiological and genetic testing.

### SUMMARY OF THE INVENTION

The invention is based on the problem of how to develop a source of brain tissue, which can be used for transplantation therapy or from which such transplants can easily be derived. This tissue must not bear any of the above mentioned disadvantages. In addition, this invention includes the problem of presenting a method for manufacturing such tissue, and the solution of the problem as described and claimed below.

This invention is based on the concept that neuronal progenitor (precursor) cells can be isolated and expanded in vitro. The proliferation of said neuronal progenitor cells can be modulated using appropriate substances, e. g. proteins, in a way that these cells become determined to differentiate completely or predominantly into a specific cell type (e.g. dopaminergic neuron) after transplantation or, in general, after making contact with an appropriate substrate such as other cells or a supporting material. Using this treatment, one can generate tissue cultures that almost substantially contain immediate precursors of specific neurons. These cultures do not include cells that give rise to immunogeneic glial cells in large enough quantities to induce any detectable immune response. Thus, the inventor can generate appropriate well-characterized tissue for transplantation with virtually unlimited supply.

Another aspect of the present invention includes isolated brain derived tissue not containing any physiologically active amounts of immunocompetent glial cells. The tissue almost substantially consists of dopaminergic neurons, cholinergic neurons, GABAergic neurons, and/or serotonergic neurons, or differentiates into these neurons.

Another aspect of the present invention includes isolated brain derived tissue not containing any physiologically active amounts of immunocompetent glial cells substantially consists of dopaminergic neurons or cells that can differentiate into dopaminergic neurons; or cholinergic neurons or cells that can differentiate into cholinergic neurons; or GABAergic striatal neurons or cells that can differentiate into GABAergic striatal neurons; or serotonergic neurons or cells that can differentiate into serotonergic neurons.

Another aspect of the present invention includes isolated brain derived tissue not containing any physiologically active amounts of immunocompetent glial cells that is derived from mammals, and especially humans. Alternatively, such isolated brain derived tissue derived from mammals, especially humans, is derived from developing immature (progenitor) cells.

Another aspect of the present invention includes a monoclonal cell line derived from mammalian, especially human, progenitor cells characterized by exclusive or predominant differentiation into neurons when exposed to differentiation promoting factors.

### BRIEF DESCRIPTION OF THE DRAWINGS

FIG. 1 is a graph showing cell proliferation of human midbrain-derived progenitor cells under normal atmospheric and reduced oxygen conditions with normal or reduced oxygen assessed by total protein content per flask.

FIG. 2 is an image of a representative phase contrast microphotograph of a cell cluster (“neurosphere”) of progenitor cells after 20 days in culture.

### DETAILED DESCRIPTION OF THE INVENTION

This invention allows the generation of tissue that substantially contains dopamineric and/or cholinergic and/or GABAergic and/or serotonergic neurons alone or any combination thereof. The percentage of such specific neurons in the tissue samples should be greater than 90%, preferably greater than 95%. Thus, the tissue does not contain other cells, e.g. glial cells, which would be physiologically relevant.

Neuronal progenitor cells from which the tissue for transplantation is derived can be isolated from embryonic or adult brain or spinal cord preparations. If an adult donor is used, neuronal progenitor cells are preferably isolated from subventricular or hippocampal brain regions. Neuronal progenitor cells are abundant in embryonic brain tissue. Thus, brain regions may be selected that normally contain the neurons of interest. Neuronal progenitor cells that differentiate into dopaminergic neurons may best be isolated from midbrain tissue. This invention, however, allows generation of different determined progenitor cells from the same pluripotent progenitor cell pool, which may also be derived from umbilical cord blood. Most efficiently, neuronal progenitor cells are prepared from human embryonic brain tissue, 3-25 weeks of gestation, preferably 5-11 weeks of gestation.

Isolation and culturing of neuronal progenitor cells from rodent brain has been reported (Daadi und Weiss, J. Neurosci 1999; Magrassi et al., Development 1998; 54:107-115; Ptak et al., Cell Transplant 1995; 4:299-310; Liepelt et al., Brain Res Dev Brain Res 1990; 51:267-278). Neuronal progenitor cells were successfully isolated from various parts of the brain. In addition, neuronal progenitor cells could also be isolated from human embryonic brain tissue (Buc-Caron, Neurobiol Dis 1995; 2:37-47; Svandsen CN et al., Exp Neurol 1997; 148:135-146; Sah et al., Nat Biotechnol 1997; 15:574-580; Chalmers-Redman et al., Neuroscience 1997; 76:1121-1128. The technique of preparation of brain tissue and isolation of neuronal progenitor cells has been adapted from these protocols.

Tissue that can be used for transplantation of patients is prepared according to the invention which includes the expansion of direct or indirectly harvested progenitor cells, partial differentiation in vitro and a selection of cells. The resulting tissue cultures differentiate into specific cell types preferably without additional application of compounds or genetic engineering.

A population of determined neuronal progenitor cells that have been selected and partially differentiated maintains the ability to perform mitosis allowing for performing subsequent proliferation steps. Partial differentiation and selection may be performed repeatedly with possible variation among individual treatments.

This invention finally allows modulation of immature pluripotent neuronal progenitor cells that become highly determined progenitor cells that will predominantly or only differentiate into a specific cell type after transplantation or in vitro differentiation.

Expansion of neuronal progenitor cells may include a variation of atmospheric oxygen content, priming, transient or non-transient expression of foreign genes, treatment with exogenous compounds especially under reduced oxygen partial pressure, or a combination of these. These individual treatments will be explained in detail below.

The selection of determined progenitor cells includes generation of clonal cell lines, which may include a variation, especially a reduction, of atmospheric oxygen.

The procedure may include selective expansion of freshly isolated progenitor cells. Proliferation of selected cells may be promoted using a modulation of atmospheric oxygen content, or by application of appropriate mitogens or by priming with exogenous compounds that stimulate differentiation, if desired each under reduced oxygen content of the atmosphere. Before or after priming cells may be subcloned, if desired under reduced oxygen partial pressure. In addition, transient expression of foreign genes may be used to promote further determination of individual clonal cell lines. The effect induced by the reduction of atmospheric oxygen content may be simulated or enhanced using conditions that exert similar effects on cell metabolism (e. g. inhibitors of mitochondrial energy production such as rotenone, MPP+ or malonate). If desired a further expansion or further partial differentiation by the methods mentioned above can be conducted.

Expansion of determined progenitor cells preferably originates from a single cell.

The success of selection of determined cell lines, which is the characterization after complete in vivo (after transplantation) or in vitro differentiation is performed using cytometric, biochemical, molecular biology, immunohistochemical and/or electrophysiological methods (see below).

Expansion using a Modulation of Atmospheric Oxygen Content:

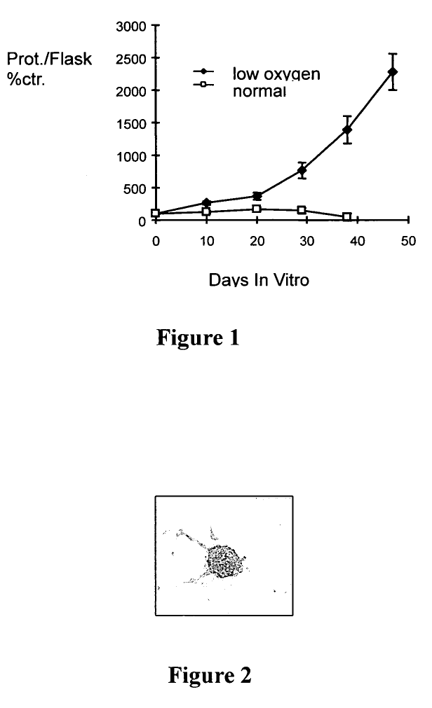

The rate of proliferation of neuronal progenitor cells can be increased using a reduction of oxygen and/or an increase of nitrogen concentrations in the incubator. In addition, these modulations of culturing conditions promote the proliferation of specific neurons (e. g. dopaminergic neurons). At present, human embryonic midbrain derived progenitor cells may only be expanded using such conditions (FIG. 1). For example, the oxygen content may be lowered from 20% (room air) to 10%, better 5% or preferably 1%, especially with a corresponding increase in nitrogen content. However, addition of other gaseous compounds is possible. The reduction of the oxygen content is performed when cells are supplemented with mitogens, which is the expansion state. As mentioned above similar effects may be obtained using inhibitors of mitochondrial respiration.

The exogenous mitogens (detailed description below) maybe used in concentrations varying from 4000 to 0.01 ng/ml, better 500 to 1/ml, preferably 100 to 2 ng/ml. Concentrations outside these ranges are not excluded.

Partial Differentiation using Priming:

Priming includes intermittent treatment of (monoclonal) neuronal progenitor cells with one or more compounds that promote differentiation in specific neurons. These compounds include e.g. growth factors, cytokines, neurotransmitters. In addition, conditioned media may be employed. These media may be derived from primary cultures containing striatal, glial or other brain cells or used to cultivate these neurons. The media contain amino acid compounds being secreted from these cells. Preferably cells of the target region of the neurons of choice are used. To generate-tissue for transplantation these media may be serum-free. These compounds are removed after a period (preferably a few hours) that allows dedifferentiation into progenitor cells that maintain their capability to perform mitosis. Said primed progenitor cells respond to a subsequent treatment with such factors more rapidly. Said primed progenitor cells may be subcloned and/or expanded. Priming may be repeated several times using identical or alternative combinations and/or concentrations of differentiation promoting compounds. The progenitor cells are partially differentiated by priming using treatment with appropriate cytokines, growth factors, hormones, neurotransmitters, transcription factors and/or gangliosides for periods of time that induce expression of tissue specific genes but do not preclude proliferation.

Priming may be performed with a variety of substances (exogenous factors). One may use combinations of cytokines and growth factors, cytokines and neurotransmitters, cytokines and hormones, cytokines and gangliosides, cytokines and conditioned media, growth factors and neurotransmitters, growth factors and hormones, growth factors and gangliosides, growth factors and conditioned media, neurotransmitters and hormones, neurotransmitters and gangliosides, neurotransmitters and conditioned media, etc.

Any of the above mentioned combinations may again be combined. Growth factors comprise one or more of the epidermal growth factor (EGF) family, preferably EGF1, EGF2, or EGF3 including α and β subgroups, transforming growth factor (TGF) α and β, LIN-3, fibroblast growth factor (FGF) 1 and 2, nerve growth factor (NGF), brain derived neurotrophic factor (BDNF), neurotrophines (NT) 3, 4, 5 and 6, insulin like growth factor (IGF) 1 and 2, glial cell line-derived neurotrophic factor (GDNF), Neurturin (NTN), Persephin (PSP), vascular endothelial growth factor (VEGF) and platelet derived growth factor (PDGF), including all members of individual families and proteins with similar mode of action.

Cytokines may include one or a combination of leukemia inhibitory factor (LIF), ciliary neurotrophic factor (CNTF), the family of interleukins (IL1-IL6), tumor necrosis factor (TNF), especially TNFα, interferons (IFN), especially IFN-α, macrophage inhibitory or stimulating factor, especially macrophage migration inhibitory factor (MIF), mitochondrial import stimulation factor (MSF) and retinoic acid.

Treatment with neurotransmitters may include one or a combination of dopamine, acetylcholine, GABA, glutamate, glycine, taurine, proline, noradrenaline, serotonin and various neuropeptides such as substance P and enkephalin.

In addition one or combinations of various hormones, especially steroid hormones or thyroid hormone, gangliosides, and their derivatives may be used.

Priming of neuronal progenitor cells in order to determine these to differentiate into dopaminergic neurons preferably includes treatment with GDNF, LIF, IL1, IL11 and/or thyroid hormone.

These exogenous compounds may be administered in concentrations ranging from 25,000 to 0.005 ng/ml, better 1000 to 0.1 ng/ml, preferably 100 to 1 ng/ml expansion media. Concentrations outside these ranges are not excluded.

Especially, IL-1 concentrations of 0.005 to 10 ng/ml, preferably 0.01 to 2 ng/ml, especially between 0.05 to 0.25 ng/ml could be used. IL-11 and LIF could be applied in concentrations of 0.01 to 100 ng/ml, preferably 0.1 to 20 ng/ml, most preferably between 0.5 to 2.5 ng/ml. GDNF could be applied in concentrations from 1 to 25,000 ng/ml, preferably 1-10 to 5,000 ng/ml, most preferably between 1-100 to 2,500 ng/ml.

These concentrations of exogenous factors may also be used when combinations of any of these are employed. However, the actual concentrations are not limited to the above mentioned ranges and may vary depending on the combination.

Partial Differentiation using Transfection:

The generation of highly determined neuronal progenitor cells may also include genetic engineering, especially in combination with priming. Using transfection with genes that are known to be crucial for the development of specific cell types, a high degree of determination may be achieved. A transient transfection is preferred since integration of plasmid DNA into the chromosomal DNA of such cells is not warranted to avoid administration of foreign genes into the host brain.

Determination to differentiate into dopaminergic neurons may be promoted via expression of members of the steroid or thyroid hormone receptors, tyrosine hydroxylase, NURR1 and/or NURR77. In addition, genes encoding for the vesicular monoamine transporter (VMAT2) or the dopamine transporter may be used. In general, all genes that play a role in the development of such neurons may be employed.

To select for neuronal progenitor cells that are determined to differentiate into cholinergic neurons, genes of the nicotinic acetylcholine receptor family, NGF receptors or cholinesterase can be used.

Progenitors determined to differentiate into GABAergic neurons may be generated via transient transfection with dopamine receptor, glutamate receptor, γ amino butyric acid transporters enkephaline and/or substance P genes.

All of the above mentioned cDNAs are known and available. Transfection may be performed using standard procedures resulting in transient or stable expression of these genes.

Generation of Monoclonal Cell Lines:

To select for determined cell lines, one may generate monoclonal cell lines. Using appropriate protocols it is possible to generate cell lines that are derived from a single cell (monoclonal cell line). Subcloning has been proven useful to minimize the heterogeneity of various cell suspensions. Using rat embryonic midbrain derived monoclonal cell lines individual clones could be identified of which 98% of the cells expressed proteins which are specific of dopaminergic neurons (tyrosine hydroxylase) following in vitro differentiation.

Subcloning may be performed using dilutions of single cell suspensions or may be aided using fluorescence-activated cell sorting (FACS) after labeling of vital cells or via enriching these cell suspensions using a magnetic column after labeling the cells with superparamagnetic beads or micromanipulation.

Subcloning with suspensions of single cells may be performed using gravity extraction of non-dissociated cells and dilution of the remaining single cells to a concentration calculated to contain only one cell per volume that is needed for plating. Cells are plated in expansion media allowing for proliferation of monoclonal cell lines. Said monoclonal cell lines will be treated and characterized as described above.

Micromanipulation may be performed to increase the yield of monoclonal determined cell lines. Viable cells will be labeled with a fluorescent marker that is specific for the respective cell population. Using fluorescent microscopy single fluorescent cells can be identified and selected with appropriate tools such as a glass capillary. These cells may then give rise to monoclonal cell lines which have a much higher potential to be determined to differentiate into the cell type of choice. Preferably mitogenic substances (see above) are added to the expansion media. Differentiation and characterization is conducted as described below. Neuronal progenitor cells that express the tyrosine hydroxylase gene may be labeled using expression of a fluorescent protein (e. g. EGFP) which is controlled by the promoter of the tyrosine hydroxylase or dopamine transporter gene. Similar approaches may be used to identify neuronal progenitor cells that already express specific proteins using the choline-acetyl-transferase promoter (cholinergic marker), glutamyl transferase (GABAergic marker) or other respective promoter elements. In addition, viable cells may also be identified using fluorescent antibodies to specific membrane localized proteins (dopamine transporter, nicotinic acetylcholine receptors, especially α, β subunits (especially α7 subunits) GABA transporter, etc.).

Similar labeling techniques may also be employed to isolate specific neuronal progenitor cells using FACS. Said labeled cells may be separated from unlabeled cells using standard FACS protocols (for review: Orfao and Ruiz-Arguelles, Clin Biochem 1996;29:5-9). These isolated cells may be expanded using polyclonal cell lines or via subcloning of monoclonal cell lines after dilution of single cell suspensions.

Magnetic isolation of determined neuronal progenitor cells may be performed using labeling of viable cells with superparamagnetic beads. These beads are commercially available (Basic microbeads—dextran coated with free amines, 50 nm, Miltenyi Biotech; Amino/Carboxy beads, 110-140 nm, Immunicon Corp.; Streptavidin/Biotin coated, Miltenyi Biotech or Immunicon Corp.).

Ligands for specific proteins (e. g. dopamine D2 receptor, dopamine transporter, nicotinic acetycholine receptors, GABA transporter, serotonine transporter, etc.) may be fused to the surface of these beads. Suspensions of individual cells may be incubated with these loaded beads. After binding of the magnetic beads to individual cells, these cells can be isolated via contact with a magnetic column. When the magnet in the column is turned off, cells that express the desired proteins can be eluted and used to generate polyclonal or monoclonal cell lines. Neuronal progenitor cells that express dopaminergic neuron specific proteins such as dopamine receptors or dopamine transporter may be identified using spiperone or benzamide derivates as ligands for dopamine D2 receptors or cocaine derivatives as ligands for the dopamine transporter. For labeling of GABAergic cells, ligands for the GABA transporter may be used. Cholinergic cells may be recognized using ligands for acetylcholine receptors.

All of the above mentioned procedures are used to isolate (subclone) cells which are highly determined to differentiate into a given cell type but maintain their ability to divide. These procedures include priming with exogenous factors that stimulate differentiation, expression of foreign genes and/or changes in atmospheric oxygen and nitrogen concentrations.

Expansion after subcloning, micromanipulation, magnetic isolation and/or FACS is always performed using identical or similar expansion media as described above.

All of the above mentioned procedures may be combined or repeated.

Isolation and Expansion of Neuronal Progenitor Cells:

1. Fetal and Adult Progenitor Cell Cultures (Expansion): Embryonic brain tissue from 5 to 12 weeks after gestation may be acquired under compliance with German Ärztekammer guidelines, German government guidelines, and the local ethics committee and appropriate consent forms were used. Samples may be collected and the forebrain and ventral mesencephalon including the subependymal region may be dissected. To confirm the origin of midbrain samples, a small amount of tissue should be processed further for primary culture and stained for tyrosine hydroxylase (TH). The tissue samples may be serially incubated with serine protease such as trypsin (50-500 mg/ml) for 30 min at 23° C. and DNAse (20-60 μg/ml) for 2-30 min at 37° C., mechanically titrated to a quasi-single cell suspension and plated into uncoated 25 cm²-flasks (0.05-10×10⁶ cells per flask) in 5 ml expansion media, supplemented with efficient concentrations of mitogens (EGF, 10-100 ng/ml and/or FGF2, 5 to 100 ng/ml or others). Cultures may be placed in a humidified incubator at 37° C. and 5% CO₂, 95% air or at lowered O₂ conditions using an O₂-sensitive electrode system. Growth factors will be supplemented every other to every second day and cultures will be passed every 10 to 20 days. Cells may be labeled with BrdU with 10 μM BrdU (Sigma) 2, 4 and 7 days after plating for 24 h. The expansion media may contain mitogens and 10%-60% F12 or 30%-60% Dulbecco's Modified Eagle's Medium (DMEM; without glucose or with various glucose concentrations), efficient concentrations of an antibiotic (50 to 250 units/ml penicillin and 50 to 250 μg/ml streptomycin). In addition, the expansion media may contain one or combinations of the following compounds: transferring, diamines, especially putrescine, sodiumselenit, gestagens, especially progesterone or similar compounds and insulin. Commercially available mixtures of supplements such as B27 (Gibco) may be used instead or in addition

2. Expansion in altered atmospheric conditions: Normal room air contains about 20% oxygen and less than 1% CO₂. Tissue culturing is usually performed in air that contains the same amount of oxygen (20%) and 1%-10%, preferably about 5% CO₂. The rate of proliferation of various neuronal progenitor cells depends on this air composition. As indicated above human midbrain derived progenitor cells and cells that differentiate into dopaminergic neurons are specifically promoted using reduced oxygen condition. Neuronal progenitor cells may, therefore, be incubated in adequate systems that allow tight and continuous control not only of CO₂ but also oxygen content and indirectly nitrogen content using a O₂-sensitive electrode system. Oxygen concentrations may vary between less than 1% and 30%. To promote proliferation of progenitor cells determined to differentiate into dopaminergic neurons 1%-5% oxygen is preferably employed.

3. Transfection of Cells with Genes: One may transfect cells during proliferation to prepare these cells for cell sorting. Plasmid DNA may be added in concentrations of 0.1-5 μg/ml, preferably 0.5-1.0 μg of plasmid DNA per ml content of tissue culture flask and appropriate amounts of commercially available transfection reagents, e. g. 3 μl per μg DNA of TransFast (Promega) solution (prepared according to the instructions of the manufacturer). This solution may be incubated at 37° C. and then added to the tissue culture flasks. To identify differentiated neurons one may incubate DNA and transfection solutions in complete differentiation media 1 hour at 37° C. Cells may be harvested, washed and resuspended in the differentiation media containing plasmid DNA and lipofectin for another hour at 37° C. before plating on precoated tissue culture dishes.

Characterization of Neuronal Progenitor Cells before and after Differentiation:

1. Measurement of Proliferation by [³)H]Thymidin Incorporation and Protein Determination: [³H]Thymidin incorporation and protein determination may be carried out according to standard procedures reported in the literature.

2. Measurement of Proliferation and Viability by Flow Cytometry: Since progenitor cells are sensitive to mechanical stress and grow in neurospheres, which may be difficult to homogenize to single cell suspension, cell count and viability check can not be performed using standard procedures. One may use electronic cell analysis (e. g. CASY® TTC system). This system is based on flow cytometry and allows for measuring cell count, whole cell volume, cell volume, and various other cell parameters.

3. Differentiation of Progenitor Cells: Cells may be differentiated in vitro by plating them onto poly-L-lysin-coated cover slips or 48 well-plates in neurobasal media (Gibco). Media may be supplemented with FCS, cytokines and/or striatal-conditioned media. The following cytokines will be used: Interleukin 1b (IL-1b), IL-11, leukemia inhibitory factor (LIF), and glial cell line-derived factor (GDNF) or other exogenous factors (described above in respect to priming). The cells are allowed to differentiate for 7 to 10 days at 37° C. in a humidified atmosphere before fixation and immunostaining.

4. Toxicological Assays: MTT assay: After incubation of the cultures with the substance of interest, 30 μl of MTT reagent (0.5 mg/ml MTT in PBS containing 10 mM HEPES) may be added to each well and incubated at 37° C. for 2 h. The medium is aspirated from each well and the culture plate dried at 37° C. for 1 h. The resulting formazan dye can be extracted with 100 μl acid-isopropanol and the absorbency measured spectrophotometrically using computer-operated immuno reader at a wavelength of 570 nm with reference at 630 nm. Wells without cells will be used as blanks and are subtracted as background from each sample. Trypan blue exclusion method: The trypan blue assay will be carried out according to standard procedures.

5. Immunocytochemistry: Cultures may be fixed using 3.7% paraformaldehyde and washed with PBS. After blocking with normal serum, primary antibody may be added and incubated over night at 4° C. The following day, primary antibody may be removed and biotinylated secondary antibody added for 1 h followed by visualization via the ABC system coupled to nickel/DAB/H₂O₂ reaction or fluorescence-conjugated antibody. All cultures may be incubated with secondary antibody without primary antibody to ensure the specificity of the reaction. All plates may be assessed for the distinct staining by an individual blinded to treatment history. For visualization of dopamine cells anti-tyrosine hydroxylase and anti-dopamine transporter antibodies may be used, for GABAergic cells antibodies against anti-GAD65 & 67, for cholinergic cells antibodies against ChAT, for glial cells anti-GFAP antibodies, for neurons anti-MAP2 and anti-β-tubulin III, for oligodendrocytes anti-O4 antibodies may be used. For anti-BrdU staining to demonstrate proliferation of the cells the method according to the manufacturer (RPN-20 kit; Amersham) may be used. For double staining (in particular anti-BrdU and TH, anti-tubuline and TH or GAD) to demonstrate specific neuronal and glial phenotypes from progenitor cell origin, immunofluorescence stained cultures may be assessed using the fluorescence microscope equipped with visual analysis system (Axiovert 135; Zeiss).

6. Transmitter High-Affinity Uptake Studies: Functional integrity of DA and GABA neurons may be evaluated by measuring the uptake of their respective tritiated neurotransmitter. After preincubation for 10 min in incubation buffer containing 100 μM pargyline, 1 mM ascorbate, and 2 mM β-alanine (and for determination of nonspecific uptake: 3 μM GBR12909 and 1 mM 2,4-diamino-n-butyric acid; DABA), 50 nM [³H]DA. [³H]choline or [³H]GABA may be added for 15 min at 37° C. Uptake may be stopped by washing the dishes with cold PBS and the remaining radioactivity in the cell lysate may be measured using liquid scintillation counting. Specific uptake may be defined as the difference between the uptake measured in the absence (total) and the uptake measured in the presence of GBR12909 and DABA (nonspecific).

7. Determination of Dopamine, Acetylcholine and GABA by HPLC: For determination of dopamine, GABA and acetylcholine a HPLC-based method may be used. Determination of acetylcholine and GABA may be performed using standard procedures.

I claim:

1. A method for generating an expandable tissue culture comprising the following steps:
(a) isolating progenitor cells from mammalian brain tissue;
(b) proliferating the progenitor cells;
(c) priming the proliferated progenitor cells by transient treatment with a differentiation-promoting factor selected from the group consisting of a cytokine, growth factor, hormone, neurotransmitter, transcription factor and ganglioside, such that the primed, proliferated progenitor cells maintain their capacity to perform mitosis and respond to subsequent treatment with the factors more rapidly than unprimed proliferated progenitor cells, if treated with a differentiation-promoting factor again;
(d) subcloning one of the primed proliferated progenitor cells; and
(e) proliferating the subcloned primed cell to produce the tissue culture.

2. The method according to claim 1, wherein one or more steps (a), (b) and (c) is repeated one or more times.

3. The method according to claim 1, wherein the subcloning step (d) is performed using one or more of a procedure selected from the group consisting of (i) final dilution; (ii) micromanipulation; (iii) fluorescence activated cell sorting; and (iv) labeling and isolation with super-paramagnetic beads.

4. The method according to claim 1, wherein one or more of steps (a) through (e) is performed in conditions of at least one of reduced oxygen content and increased nitrogen content.

5. The method according to claim 4, wherein the oxygen content is reduced at least to 10%.

6. The method according to claim 4, wherein the oxygen content is reduced at least to 5%.

7. The method according to claim 1, wherein one or more of steps (a) through (e) is performed in conditions that simulate reduced oxygen content.

8. The method according to claim 7, wherein the conditions that simulate reduced oxygen content include application of a compound that inhibits mitochondrial respiration.

9. The method according to claim 1, wherein step (c) comprises priming the proliferated progenitor cells by transient treatment with a differentiation-promoting factor for a time that induces expression of tissue-specific genes but does not preclude proliferation.

10. The method according to claim 1, wherein the differentiation-promoting factor is one or a combination of a growth factor selected from the group consisting of EGF, FGF, GNDF, TGF α and β, LIN-3-protein, NGF, BDNF, NT, PDNF, IGF and VEGF.

11. The method according to claim 1, wherein the differentiation-promoting factor is one or a combination of a cytokine selected from following group consisting of LIF, CNTF, any one of interleukin IL1 to IL16, interferon, MIF, MSF, and retinoic acid.

12. The method according to claim 1, wherein the differentiation-promoting factor is one or a combination of a neurotransmitter selected from the group consisting of dopamine, acetylcholine, GABA, glutamate, glycine, taurine, proline, noradrenaline, serotonin, substance P. and enkephalin.

13. The method according to claim 1, wherein priming step (c) is performed on cells subcloned from monoclonal progenitor cell lines.

14. A method for generating an expandable tissue culture comprising the following steps:
(a) isolating progenitor cells from mammalian brain tissue;
(b) proliferating the progenitor cells;
(c) transfecting the proliferated progenitor cells with a gene crucial for development of a specific cell type, wherein the gene is selected from the group consisting of a gene for a steroid receptor, hormone receptor, tyrosine hydroxylase, NURR1, NURR77, VMAT2, dopamine transporter, nicotinic acetylcholine receptor, NGF receptor, cholinesterase, dopamine receptor, glutamate receptor, gamma-amino butyric transporter, enkephalin and substance P gene, wherein the transfected, proliferated progenitor cells maintain their capacity to perform mitosis and express the genes with which they are transfected;
(d) subcloning one of the transfected proliferated progenitor cells; and
(e) proliferating the subcloned transfected cell.

15. The method according claim 14, wherein the transfection is transient.

16. The method according to claim 14, further comprising priming the proliferating progenitor cells before or after the transfecting step (c).

17. The method according to claim 16, wherein step (c) comprises priming the proliferated progenitor cells by transient treatment with a differentiation-promoting factor for a time that induces expression of tissue-specific genes but does not preclude proliferation.

18. Tissue culture generated according to claim 1.

19. Tissue culture generated according to claim 14.
